(19) United States
(12) Patent Application Publication
(10) Pub. No.: US 2026/0137681 A1
(43) Pub. Date: May 21, 2026

(54) EPIDERMAL GROWTH FACTOR RECEPTOR TYROSINE KINASE INHIBITORS IN COMBINATION WITH HGF-RECEPTOR INHIBITORS FOR THE TREATMENT OF CANCER

(71) Applicant: AstraZeneca AB, Sodertalje (SE)

(72) Inventors: Ryan HARTMAIER, Wilmington, DE (US); Gina DANGELO, Wilmington, DE (US)

(21) Appl. No.: 18/992,063

(22) PCT Filed: Jul. 7, 2023
(86) PCT No.: PCT/EP2023/068862

§ 371 Date: Jan. 7, 2025

Related U.S. Application Data
(60) Provisional application No. 63/367,949, filed on Jul. 8, 2022.
(60) Provisional application No. 63/370,501, filed on Aug. 5, 2022.

(51) Int. Cl. A61K 31/506; A61K 31/4985; A61K 31/517; A61K 31/53; A61K 31/5377; A61P 35/00

(52) U.S. Cl. CPC A61K 31/506; A61K 31/4985; A61K 31/517; A61K 31/53; A61K 31/5377; A61P 35/00

(57) ABSTRACT

The specification relates to epidermal growth factor receptor (EGFR) tyrosine kinase inhibitors (TKIs) for use in the treatment of cancer, wherein the EGFR TKI is administered in combination with an inhibitor of c-MET.

### RELATED APPLICATIONS

This application is a U.S. National Stage Application of International Application No. PCT/EP2023/068862, filed Jul. 7, 2023, which claims benefit of priority to U.S. Provisional Patent Application No. 63/367,949, filed Jul. 8, 2022, and US Provisional Patent Application No. 63/370,01, filed Aug. 5, 2022, the contents of each of which are hereby incorporated by reference in its entirety for all purposes.

### FIELD

The specification relates to an Epidermal Growth Factor Receptor (EGFR) Tyrosine Kinase Inhibitor (TKI) for use in the treatment of cancer (for example non-small cell lung cancer [NSCLC]), wherein the EGFR TKI is administered in combination with an inhibitor of c-MET (also known as HGF-receptor, or mesenchymal epithelial transition factor).

### BACKGROUND

The discovery of activating mutations in the epidermal growth factor receptor (EGFR) has revolutionized the treatment of the disease. In 2004 it was reported that activating mutations in exons 18-21 of EGFR correlated with a response to EGFR-TKI therapy in NSCLC and it is estimated that these mutations are prevalent in approximately 10-16% of NSCLC human patients in the United States and Europe, and in approximately 30-50% of NSCLC human patients in Asia. Two of the most significant EGFR activating mutations are exon 19 deletions and missense mutations in exon 21. Exon 19 deletions account for approximately 45% of known EGFR mutations. The missense mutations in exon 21 account for approximately 39-45% of known EGFR mutations, of which the substitution mutation L858R accounts for approximately 39% of the total mutations in exon 21.

There are 3 generations of EGFR TKIs: first-generation EGFR TKIs (e.g., gefitinib, erlotinib) are ATP-competitive inhibitors; second-generation EGFR TKIs (e.g., afatinib, dacomitinib) are irreversible inhibitors that bind covalently to EGFR; and third-generation EGFR TKIs (e.g., osimertinib) are designed to target the T790M resistance mutation and EGFR sensitizing mutations more selectively than wild-type EGFR. All these TKIs are effective in patients with NSCLC whose tumours harbour the in-frame deletions in exon 19 and/or the L858R point mutation in exon 21. These two mutations represent approximatively 90% of all EGFR mutations. In approximately 50% of patients, resistance to first- and second-generation EGFR TKI is mediated by the acquisition of the ‘gatekeeper’ mutation T790M. Currently, osimertinib is the only third-generation EGFR TKI approved in the US that is active against exon 19 deletions and L858R mutation, regardless of the presence of T790M mutation. However, even patients treated with osimertinib ultimately progress, predominantly due to the development of acquired resistance resulting from other resistance mechanisms. As such, there remains a need to develop new therapies for the treatment of NSCLC, especially for patients whose disease has progressed following treatment with an EGFR TKI.

Amplification of the MET gene has been reported as a secondary oncogenic event in 5% to 22% of EGFRm+ NSCLC with acquired resistance to EGFR TKIs. MET is a transmembrane receptor tyrosine kinase that can be activated by protein overexpression, increased expression of its ligand HGF, MET mutations, gene amplification, and exon 14 skipping. ctDNA data from two osimertinib clinical studies (AURA3 [NCT02151981] which investigated osimertinib or platinum-pemetrexed in patients with EGFR T790M+ lung cancer, and FLAURA [NCT02296125], which investigated osimertinib versus standard EGFR TKIs [gefitinib or erlotinib] in patients with untreated EGFRm+ advanced NSCLC) revealed acquired MET amplifications following disease progression on osimertinib in 15% to 19% of patients tested. MET overexpression as well as amplification in tumor tissue is detected in the acquired EGFR TKI resistance setting. However, despite nonclinical models suggesting that the use of a MET TKI in combination with an EGFR TKI may overcome MET-driven resistance, there is no approved therapy specifically indicated for the treatment of patients with tumors positive for MET amplification and/or overexpression and no established standard of care specifically for patients who have developed MET-driven resistance after prior EGFR TKI therapy (including osimertinib).

The absence of a therapy for patients who have developed MET-driven resistance after prior treatment with EGFR TKIs may be due to uncertainty over how to identify the patients most likely to benefit from MET TKI and EGFR TKI combination therapy. The prevalence of MET amplification and overexpression depends on the sample type, detection method and assay cut-off used. Several clinical Phase1b/2 studies (e.g. NCT02143466, NCT01610336 and NCT01982955) have investigated the efficacy of a MET TKI in combination with an EGFR TKI in MET-amplified and/or overexpressed. However, across these studies, different amplification or overexpression assay cut-offs were used for patient selection and in NCT01610336 cut-offs were even changed during the study. Moreover, there was considerable variability in objective response rate (ORR), thus casting doubt on the suitability of MET amplification and overexpression as a predictor of clinical benefit. Accordingly, there remains a considerable unmet medical need for a means to identify the EGFRm+ NSCLC patients that are most likely to benefit from MET TKI and EGFR TKI combination therapy.

### SUMMARY

The present specification enables the identification of EGFRm+ NSCLC patients that are most likely to benefit from MET TKI and EGFR TKI combination therapy using defined MET amplification and/or overexpression assay cut-offs. Without being bound by theory, it has been discovered that clinical benefit is not determined simply by the presence of MET amplification and/or overexpression, but by the degree to which such MET amplification and/or overexpression is present. Specifically, a high or very high level of MET amplification and/or overexpression is predictive of clinical benefit. Unexpectedly, it has been found that high or very high levels of MET amplification and/or overexpression are sufficiently prevalent in EGFRm NSCLC following progression on third-generation EGFR-TKIs to enable these biomarkers to be used to select the EGFRm+ NSCLC patients that are most likely to benefit from MET TKI and EGFR TKI combination therapy.

In an embodiment there is provided an EGFR TKI for use in the treatment of cancer in a human patient, wherein the EGFRTKI is administered in combination with an inhibitor of c-MET and wherein said cancer has a high or very high level of MET amplification and/or overexpression.

In an embodiment there is provided an inhibitor of c-MET for use in the treatment of cancer in a human patient, wherein the inhibitor of c-MET is administered in combination with an EGFR TKI and wherein said cancer has a high or very high level of MET amplification and/or overexpression.

In an embodiment there is provided an EGFR TKI for use in the treatment of cancer in a human patient, wherein the EGFR TKI is administered in combination with an inhibitor of c-MET, characterised in that said cancer has been found to have a high or very high level of MET amplification and/or overexpression prior to the administration of the EGFR TKI in combination with the inhibitor of c-MET.

In an embodiment there is provided an inhibitor of c-MET for use in the treatment of cancer in a human patient, wherein the inhibitor of c-MET is administered in combination with an EGFR TKI, characterised in that said cancer has been found to have a high or very high level of MET amplification and/or overexpression prior to the administration of the inhibitor of c-MET in combination with the EGFR TKI.

In an embodiment there is provided a method of treating cancer in a human patient in need of such a treatment comprising administering to the human patient a therapeutically effective amount of an EGFR TKI, wherein the EGFR TKI is administered in combination with a therapeutically effective amount of an inhibitor of c-MET and wherein said cancer has a high or very high level of MET amplification and/or overexpression.

In an embodiment there is provided a method of treating cancer in a human patient in need of such a treatment comprising administering to the human patient a therapeutically effective amount of an inhibitor of c-MET, wherein the inhibitor of c-MET is administered in combination with a therapeutically effective amount of an EGFR TKI and wherein said cancer has a high or very high level of MET amplification and/or overexpression.

In an embodiment there is provided a method of treating cancer in a human patient in need of such a treatment comprising administering to the human patient a therapeutically effective amount of an EGFR TKI, wherein the EGFR TKI is administered in combination with a therapeutically effective amount of an inhibitor of c-MET and characterised in that said cancer has been found to have a high or very high level of MET amplification and/or overexpression prior to the administration of the EGFR TKI in combination with the inhibitor of c-MET.

In an embodiment there is provided a method of treating cancer in a human patient in need of such a treatment comprising administering to the human patient a therapeutically effective amount of an inhibitor of c-MET, wherein the inhibitor of c-MET is administered in combination with a therapeutically effective amount of an EGFR TKI and characterised in that said cancer has been found to have a high or very high level of MET amplification and/or overexpression prior to the administration of the inhibitor of c-MET in combination with the EGFR TKI.

In an embodiment there is provided a method of treating cancer in human patients in need of such a treatment comprising: i) identifying patients whose cancers have a high or very high level of MET amplification and/or overexpression; and ii) administering to said identified patients a therapeutically effective amount of an EGFR TKI, wherein the EGFR TKI is administered in combination with a therapeutically effective amount of an inhibitor of c-MET.

In an embodiment there is provided a method of treating cancer in human patients in need of such a treatment comprising: i) identifying patients whose cancers have a high or very high level of MET amplification and/or overexpression; and ii) administering to said identified patients a therapeutically effective amount of an inhibitor of c-MET, wherein the inhibitor of c-MET is administered in combination with a therapeutically effective amount of an EGFR TKI.

In an embodiment, there is provided the use of an EGFR TKI in the manufacture of a medicament for the treatment of cancer in a human patient, wherein the EGFR TKI is administered in combination with an inhibitor of c-MET and wherein said cancer has a high or very high level of MET amplification and/or overexpression.

In an embodiment, there is provided the use of an inhibitor of c-MET in the manufacture of a medicament for the treatment of cancer in a human patient, wherein the inhibitor of c-MET is administered in combination with an EGFR TKI and wherein said cancer has a high or very high level of MET amplification and/or overexpression.

In an embodiment, there is provided the use of an EGFR TKI in the manufacture of a medicament for the treatment of cancer in a human patient, wherein the EGFR TKI is administered in combination with an inhibitor of c-MET and characterised in that said cancer has been found to have a high or very high level of MET amplification and/or overexpression prior to the administration of the EGFR TKI in combination with the inhibitor of c-MET.

In an embodiment, there is provided the use of an inhibitor of c-MET in the manufacture of a medicament for the treatment of cancer in a human patient, wherein the inhibitor of c-MET is administered in combination with an EGFR TKI and characterised in that said cancer has been found to have a high or very high level of MET amplification and/or overexpression prior to the administration of the inhibitor of c-MET in combination with the EGFR TKI.

In an embodiment, there is provided a method of extending progression-free survival (PFS) in a patient with cancer, the method comprising administering to the human patient a therapeutically effective amount of an EGFR TKI, wherein the EGFR TKI is administered in combination with a therapeutically effective amount of an inhibitor of c-MET and wherein said cancer has a high or very high level of MET amplification and/or overexpression.

In an embodiment, there is provided a method of increasing the ORR in a patient with cancer, the method comprising administering to the human patient a therapeutically effective amount of an EGFR TKI, wherein the EGFR TKI is administered in combination with a therapeutically effective amount of an inhibitor of c-MET and wherein said cancer has a high or very high level of MET amplification and/or overexpression.

In an embodiment, there is provided a method of extending median progression-free survival (PFS) in a patient with cancer, the method comprising administering to the human patient a therapeutically effective amount of an EGFR TKI, wherein the EGFR TKI is administered in combination with a therapeutically effective amount of an inhibitor of c-MET and wherein said cancer has a high or very high level of MET amplification and/or overexpression.

The terms “treat,” “treating,” and “treatment” refer to at least partially alleviating, inhibiting, preventing and/or ameliorating a condition, disorder, or disease, such as lung cancer. The term “treatment of cancer” includes both in vitro and in vivo treatments, including in warm-blooded animals such as humans. The effectiveness of treatment of cancer can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging survival. Treatments can also be assessed with regard to the nature and extent of side effects associated with the treatment. Furthermore, effectiveness can be assessed with regard to biomarkers, such as levels of expression or phosphorylation of proteins known to be associated with particular biological phenomena. Other assessments of effectiveness are known to those of skill in the art.

The phrase “in combination with” and similar terms (including “a combination of”) encompass administration of two or more active pharmaceutical ingredients to a subject and include simultaneous administration in separate compositions, administration at different times in separate compositions, or administration in a composition in which two or more active pharmaceutical ingredients are present.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g. the amount of apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

In a further embodiment, there is provided a pharmaceutical composition comprising an EGFR TKI, an inhibitor of c-MET and a pharmaceutically acceptable excipient.

The term “pharmaceutically acceptable” is used to specify that an object (for example a salt, dosage form or excipient [such as a diluent or carrier]) is suitable for use in patients. An example list of pharmaceutically acceptable salts can be found in the “Handbook of Pharmaceutical Salts: Properties, Selection and Use”, P. H. Stahl and C. G. Wermuth, editors, Weinheim/Zurich:Wiley-VCH/VFiCA, 2002 or subsequent editions.

Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminium. Organic bases from which salts can be derived include, for example, primary, secondary, and tertiary amines, substituted amines including naturally occurring substituted amines, cyclic amines and basic ion exchange resins. Examples include isopropylamine, trimethylamine, diethylamine, triethylamine, tripropylamine, and ethanolamine.

### DESCRIPTION OF FIGURES

FIG. 1: Kaplan-Meier plots showing progression free survival (PFS) in patients based on MET amplification and/or overexpression status.

FIG. 2: Waterfall plot showing best percentage change in target lesion in patients with a high or very high level of MET amplification and/or overexpression.

### DETAILED DESCRIPTION

### EGFR Mutation Positive NSCLC, Patient Selection, Dosing and Diagnostic Methods

In embodiments, the cancer is lung cancer, such as non-small cell lung cancer (NSCLC).

In embodiments, the NSCLC is EGFR mutation-positive NSCLC.

In embodiments, the EGFR mutation-positive NSCLC comprises activating mutations in EGFR. In further embodiments, the EGFR mutation-positive NSCLC comprises non-resistant mutations. In further embodiments, the activating mutations in EGFR comprise activating mutations in exons 18-21. In further embodiments, the activating mutations in EGFR comprise exon 19 deletions or missense mutations in exon 21. In further embodiments, the activating mutations in EGFR comprise exon 19 deletions or L858R substitution mutations. In further embodiments, the mutations in EGFR comprise a T790M mutation. In embodiments, the EGFR mutation-positive NSCLC is a locally advanced EGFR mutation-positive NSCLC. In embodiments, the EGFR mutation-positive NSCLC is a metastatic EGFR mutation-positive NSCLC. In embodiments, the EGFR mutation-positive NSCLC is not amenable to curative surgery or radiotherapy.

There are numerous methods to detect EGFR activating mutations, of which the skilled person will be aware. Tests provided by a Clinical Laboratory Improvement Amendments (CLIA) certified laboratory and/or tests that have been approved by a regulatory body, such as the US Food and Drug Administration (FDA), the European Medicines Agency (EMA) or the Chinese National Medical Products Administration (NMPA), are suitable for use in these methods. These include both tumour tissue and plasma based diagnostic methods. In general, the EGFR mutation status is first assessed using a tumour tissue biopsy sample derived from the human patient. If a tumour sample is unavailable, or if the tumour sample is negative, the EGFR mutation status may be assessed using a plasma sample. A particular example of a suitable diagnostic test to detect EGFR mutations, and in particular to detect exon 19 deletions, L858R substitution mutations and theT790M mutation, is the Cobas™ EGFR Mutation Test v2 (Roche Molecular Diagnostics). Other examples of suitable diagnostic tests include the FoundationOne CDx (Foundation Medicine) which can detect activating and resistance mutations in tissue samples; the Guardant360 CDx (Guardant Health) which can detect activating and resistance mutations in plasma samples; and the FoundationOne Liquid CDx (Foundation Medicine) which can detect activating mutations in plasma samples.

In embodiments, therefore, the EGFR mutation-positive NSCLC comprises activating mutations in EGFR (such as activating mutations in exons 18-21, for example exon 19 deletions, missense mutations in exon 21, and L858R substitution mutations; and resistance mutations such as the T790M mutation), wherein the EGFR mutation status of the human patient has been determined using an appropriate diagnostic test. In further embodiments, the EGFR mutation status has been determined using a tumour tissue sample. In further embodiments, the EGFR mutation status has been determined using a plasma sample. In further embodiments, the diagnostic method uses an FDA-approved test and/or a test provided by a CLIA certified laboratory. In further embodiments, the diagnostic method uses the Cobas™ EGFR Mutation Test (v1 or v2) or the FoundationOne CDx or the Guardant360 CDx or the FoundationOne Liquid CDx.

In embodiments, the human patient is an EGFR TKI-naïve human patient. In embodiments, the human patient has previously received EGFR TKI treatment. In embodiments the human patient has previously been treated with a third-generation EGFR TKI. In embodiments the human patient has previously been treated with osimertinib or a pharmaceutically acceptable salt thereof. In embodiments, the human patient has developed EGFR T790M mutation-positive NSCLC.

A high or very high level of MET amplification and/or overexpression in tumours can be detected using various techniques known to those in the art. For example, by applying fluorescence in situ hybridization assay (FISH) and/or an immunohistochemistry assay (IHC) to tumour tissue samples. MET amplification may also be detected by applying next generation sequencing (NGS) to plasma and/or tumour samples. In embodiments, the cancer has a high or very high level of MET amplification determined by NGS. In embodiments, the cancer has a high or very high level of MET amplification defined by ≥5 copies of MET over tumour ploidy by NGS (NGS5+).

In embodiments, the cancer has a high or very high level of MET amplification and/or overexpression determined by FISH and/or IHC. In embodiments, the cancer has a high or very high level of MET amplification and/or overexpression determined by FISH and/or IHC using a test approved by a regulatory body and/or a test provided by a CLIA certified laboratory. Relevant regulatory bodies include the EMA, the FDA and the NMPA. In embodiments, the cancer has a high or very high level of MET amplification and/or overexpression determined by FISH and/or IHC using an FDA-approved test and/or a test provided by a CLIA certified laboratory.

In embodiments, the cancer has a high or very high level of MET amplification and/or overexpression defined by MET gene copy number ≥6 by FISH (FISH6+) and/or 60% tumor cells with strong (3+) membrane and/or cytoplasmic staining intensity by IHC (IHC60+); or defined by MET gene copy number 7 by FISH (FISH7+), and/or 70% tumor cells with strong (3+) membrane and/or cytoplasmic staining intensity by IHC (IHC70+); or defined by MET gene copy number 8 by FISH (FISH8+), and/or 80% tumor cells with strong (3+) membrane and/or cytoplasmic staining intensity by IHC (IHC80+); or defined by MET gene copy number 9 by FISH (FISH9+), and/or 90% tumor cells with strong (3+) membrane and/or cytoplasmic staining intensity by IHC (IHC90+); or defined by MET gene copy number 10 by FISH (FISH10+), and/or 90% tumor cells with strong (3+) membrane and/or cytoplasmic staining intensity by IHC (IHC90+). In embodiments, the cancer has a high or very high level of MET amplification and/or overexpression defined by FISH6+, FISH7+, FISH8+, FISH9+ or FISH10+. In embodiments, the cancer has a high or very high level of MET amplification and/or overexpression defined by FISH10+. In embodiments, the cancer has a high or very high level of MET amplification and/or overexpression defined by IHC60+, IHC70+, IHC80+ or IHC90+. In embodiments, the cancer has a high or very high level of MET amplification and/or overexpression defined by IHC90+.

In embodiments, the cancer is EGFR mutation-positive NSCLC and has a high or very high level of MET amplification and/or overexpression defined by FISH10+ and/or IHC90+. In embodiments, the cancer is EGFR mutation-positive NSCLC and has a high or very high level of MET amplification and/or overexpression defined by FISH10+. In embodiments, the cancer is EGFR mutation-positive NSCLC and has a high or very high level of MET amplification and/or overexpression defined by IHC90+.

In embodiments, the cancer has been found to have a high or very high level of MET amplification and/or overexpression prior to the administration of the combination of an EGFR TKI and an inhibitor of c-MET. There are numerous FISH and/or IHC assays available to detect MET amplification and/or overexpression, of which the skilled person will be aware. In embodiments, the assays may be FDA-approved and/or provided by a CLIA certified laboratory. An example of a suitable FISH assay is the Vysis MET FISH Probe Kit (Abbott Molecular Inc., Des Plaines, IL) and an example of a suitable IHC assay is the VENTANA MET (SP44) RxDx Assay (Ventana Medical Systems, Inc., Tucson, Arizona). An example of a suitable NGS assay is F1CDx (Foundation Medicine, Cambridge, MA).

This specification discloses a combination of an EGFR TKI and an inhibitor of c-MET as a first-line (1 L) treatment (i.e. in EGFR TKI-naïve patients); as a second-line (2 L) treatment (i.e. in patients who have previously received one line of EGFR TKI treatment); and as a third-line or fourth-line (3-4 L) treatment (i.e. in patients who have previously received one line of EGFR TKI treatment with subsequent chemotherapy or in patients who have previously received two or more lines of EGFR TKI treatment with or without subsequent chemotherapy).

In embodiments, patients may have received 1, 2 or 3 lines of prior therapy, which must include an EGFR TKI as one of the prior lines of therapy, but could also include other EGFR TKIs, chemotherapy, or chemotherapy in combination with an immune-oncology (10) agent in the metastatic setting. In embodiments, the patient has received a third-generation EGFR TKI as one of the prior lines of therapy. In embodiments, the patient has received osimertinib as one of the prior lines of therapy. In embodiments, the patient has received a third-generation EGFR TKI as the most recent prior line of therapy. In embodiments, the patient has received osimertinib as the most recent prior line of therapy. In embodiments, the EGFR TKI and the inhibitor of c-MET are both administered once-daily (QD). In embodiments, the EGFR TKI is administered once-daily (QD) and the inhibitor of c-MET is administered twice-daily (BID).

In embodiments, the treatment provides an ORR of at least 35%, at least 40%, at least 45%, at least 50%, at least 55% or at least 60%.

In embodiments, the treatment provides a median PFS of at least 5.5 months, at least 6 months, at least 6.5 months, at least 7 months or at least 7.5 months.

### EGFR TKIs

Third-generation EGFR TKIs are inhibitors of EGFR bearing activating mutations that also significantly inhibit EGFR bearing the T790M mutation and do not significantly inhibit wild-type EGFR. Examples of third-generation TKIs include compounds of Formula (I), osimertinib, AZD3759 (zorifertinib), lazertinib, nazartinib (EGF816), C01686 (rociletinib), HM61713 (olmutinib), ASP8273 (naquotinib), PF-06747775 (mavelertinib), avitinib (abivertinib), alflutinib (AST2818), CX-101 (olafertinib; RX-518), aumolertinib (HS-10296; almonertinib) and BPI-7711 (rezivertinib).

In an embodiment, the EGFR TKI is a third-generation EGFR TKI. In a further embodiment, the third-generation EGFR TKI is a compound of Formula (I), as defined below. In a further embodiment, the third-generation EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, CX-101 or a pharmaceutically acceptable salt thereof, HS-10296 or a pharmaceutically acceptable salt thereof and BPI-7711 or a pharmaceutically acceptable salt thereof. In a further embodiment, the third generation EGFR TKI is osimertinib or a pharmaceutically acceptable salt thereof.

### Compounds of Formula (I)

In an embodiment, the EGFR TKI is a compound of Formula (I):

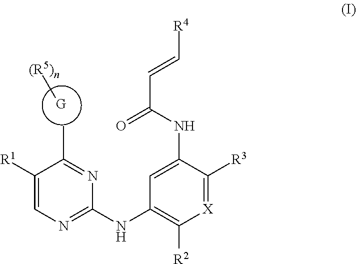

wherein:
G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;
R¹ is selected from hydrogen, fluoro, chloro, methyl and cyano;
R² is selected from methoxy, trifluoromethoxy, ethoxy, 2,2,2-trifluoroethoxy and methyl;
R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;
R⁴ is selected from hydrogen, 1-piperidinomethyl and N,N-dimethylaminomethyl;
R⁵ is independently selected from methyl, ethyl, propyl, 2,2-difluoroethyl, 2,2,2-trifluoroethyl, fluoro, chloro and cyclopropyl;
X is CH or N; and
n is 0, 1 or 2;
or a pharmaceutically acceptable salt thereof.

In a further embodiment there is provided a compound of Formula (I), as defined above, wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from[2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

Examples of compounds of Formula (I) include those described in WO 2013/014448, WO 2015/175632, WO 2016/054987, WO 2016/015453, WO 2016/094821, WO 2016/070816 and WO 2016/173438.

### Osimertinib and Pharmaceutical Compositions Thereof

### Osimertinib has the Following Chemical Structure:

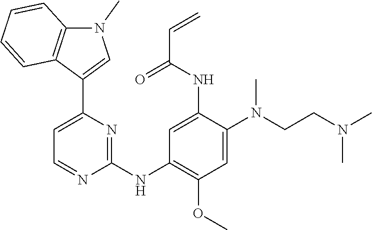 [SMILES: C=CC(=O)Nc1cc(Nc2nccc(-c3cn(C)c4ccccc34)n2)c(OC)cc1N(C)CCN(C)C]

The free base of osimertinib is known by the chemical name: N-(2-{2-dimethylamino ethyl-methylamino}-4-methoxy-5-{[4-(1-methylindol-3-yl)pyrimidin-2-yl]amino}phenyl) prop-2-enamide.

Osimertinib is described in WO 2013/014448. Osimertinib is also known as AZD9291.

Osimertinib may be found in the form of the mesylate salt: N-(2-{2-dimethylamino ethyl-methylamino}-4-methoxy-5-{[4-(1-methylindol-3-yl)pyrimidin-2-yl]amino}phenyl) prop-2-enamide mesylate salt. Osimertinib mesylate is also known as TAGRISSO™.

Osimertinib mesylate is currently approved as an oral once daily tablet formulation, at a dose of 80 mg (expressed as free base, equivalent to 95.4 mg osimertinib mesylate), for the treatment of metastatic EGFR T790M mutation positive NSCLC patients. A 40 mg oral once daily tablet formulation (expressed as free base, equivalent to 47.7 mg osimertinib mesylate) is available should dose modification be required. The tablet core comprises pharmaceutical diluents (such as mannitol and microcrystalline cellulose), disintegrants (such as low-substituted hydroxypropyl cellulose) and lubricants (such as sodium stearyl fumarate). The tablet formulation is described in WO 2015/101791.

In an embodiment, therefore, osimertinib, or a pharmaceutically acceptable salt thereof, is in the form of the mesylate salt, i.e. N-(2-{2-dimethylamino ethyl-methylamino}-4-methoxy-5-{[4-(1-methylindol-3-yl)pyrimidin-2-yl]amino}phenyl) prop-2-enamide mesylate salt.

In an embodiment, osimertinib, or a pharmaceutically acceptable salt thereof, is administered once-daily. In a further embodiment, osimertinib mesylate is administered once-daily.

In an embodiment, the total daily dose of osimertinib is about 80 mg. In a further embodiment, the total daily dose of osimertinib mesylate is about 95.4 mg.

In an embodiment, the total daily dose of osimertinib is about 40 mg. In a further embodiment, the total daily dose of osimertinib mesylate is about 47.7 mg.

In an embodiment, osimertinib, or a pharmaceutically acceptable salt thereof, is in tablet form.

In an embodiment, osimertinib, or a pharmaceutically acceptable salt thereof, is administered in the form of a pharmaceutical composition comprising one or more pharmaceutically acceptable excipients (for example a diluent or carrier). In a further embodiment, the composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as sodium stearyl fumarate).

In an embodiment, the composition is in the form of a tablet, wherein the tablet core comprises: (a) from 2 to 70 parts of osimertinib, or a pharmaceutically acceptable salt thereof; (b) from 5 to 96 parts of two or more pharmaceutical diluents; (c) from 2 to 15 parts of one or more pharmaceutical disintegrants; and (d) from 0.5 to 3 parts of one or more pharmaceutical lubricants; and wherein all parts are by weight and the sum of the parts (a)+(b)+(c)+(d)=100.

In an embodiment, the composition is in the form of a tablet, wherein the tablet core comprises: (a) from 7 to 25 parts of osimertinib, or a pharmaceutically acceptable salt thereof; (b) from 55 to 85 parts of two or more pharmaceutical diluents, wherein the pharmaceutical diluents comprise microcrystalline cellulose and mannitol; (c) from 2 to 8 parts of pharmaceutical disintegrant, wherein the pharmaceutical disintegrant comprises low-substituted hydroxypropyl cellulose; (d) from 1.5 to 2.5 parts of pharmaceutical lubricant, wherein the pharmaceutical lubricant comprises sodium stearyl fumarate; and wherein all parts are by weight and the sum of the parts (a)+(b)+(c)+(d)=100.

In an embodiment, the composition is in the form of a tablet, wherein the tablet core comprises: (a) about 19 parts of osimertinib mesylate; (b) about 59 parts of mannitol; (c) about 15 parts of microcrystalline cellulose; (d) about 5 parts of low-substituted hydroxypropyl cellulose; and (e) about 2 parts of sodium stearyl fumarate; and wherein all parts are by weight and the sum of the parts (a)+(b)+(c)+(d)+(e)=100.

AZD3759 (zorifertinib)

### AZD3759 has the Following Chemical Structure:

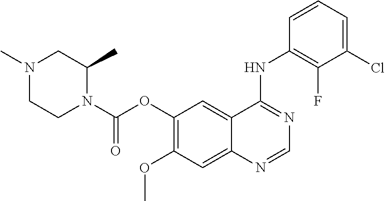 [SMILES: COc1cc2ncnc(Nc3cccc(Cl)c3F)c2cc1OC(=O)N1CCN(C)C[C@H]1C]

The free base of AZD3759 is known by the chemical name: 4-[(3-chloro-2-fluorophenyl)amino]-7-methoxy-6-quinazolinyl (2R)-2,4-dimethyl-1-piperazinecarboxylate. AZD3759 is described in WO 2014/135876.

In an embodiment, AZD3759, or a pharmaceutically acceptable salt thereof, is administered twice-daily.

In a further embodiment, AZD3759 is administered twice-daily.

In an embodiment, the total daily dose of AZD3759 is about 400 mg. In a further embodiment, about 200 mg of AZD3759 is administered twice a day.

### Lazertinib

### Lazertinib has the Following Chemical Structure:

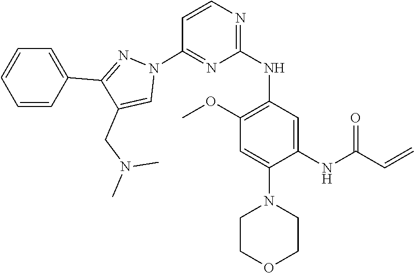 [SMILES: C=CC(=O)Nc1cc(Nc2nccc(-n3cc(CN(C)C)c(-c4ccccc4)n3)n2)c(OC)cc1N1CCOCC1]

The free base of lazertinib is known by the chemical name N-{5-[(4-{4-[(dimethylamino)methyl]-3-phenyl-1H-pyrazol-1-yl}-2-pyrimidinyl)amino]-4-methoxy-2-(4-morpholinyl)phenyl}acrylamide.

Lazertinib is described in WO 2016/060443. Lazertinib is also known by the names YH25448 and GNS-1480.

In an embodiment, lazertinib, or a pharmaceutically acceptable salt thereof, is administered once-daily.

In a further embodiment, lazertinib is administered once-daily.

In an embodiment, the total daily dose of lazertinib is about 20 to 320 mg.

In an embodiment, the total daily dose of lazertinib is about 240 mg.

### Avitinib

### Avitinib has the Following Chemical Structure:

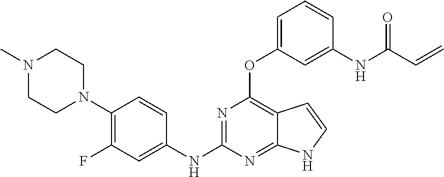 [SMILES: C=CC(=O)Nc1cccc(Oc2nc(Nc3ccc(N4CCN(C)CC4)c(F)c3)nc3[nH]ccc23)c1]

The free base of avitinib is known by the chemical name: N-(3-((2-((3-fluoro-4-(4-methylpiperazin-1-yl)phenyl)amino)-7H-pyrrolo(2,3-d)pyrimidin-4-yl)oxy)phenyl)prop-2-enamide. Avitinib is disclosed in US2014038940. Avitinib is also known as abivertinib.

In an embodiment, avitinib or a pharmaceutically acceptable salt thereof, is administered twice daily. In a further embodiment, avitinib maleate is administered twice daily.

In an embodiment, the total daily dose of avitinib maleate is about 600 mg.

### Alflutinib

### Alflutinib has the Following Chemical Structure:

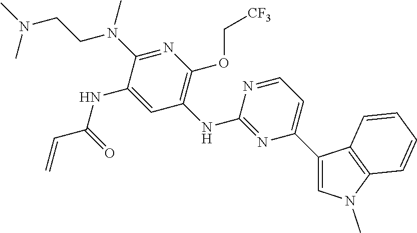 [SMILES: C=CC(=O)Nc1cc(Nc2nccc(-c3cn(C)c4ccccc34)n2)c(OCC(F)(F)F)nc1N(C)CCN(C)C]

The free base of alflutinib is known by the chemical name:

N-{2-{[2-(dimethylamino)ethyl](methyl)amino}-6-(2,2,2-trifluoroethoxyl)-5-{[4-(1-methyl-1H-indol-3-yl)pyrimidin-2-yl]amino}pyridin-3-yl}acrylamide. Alflutinib is disclosed in WO 2016/15453. Alflutinib is also known as AST2818.

In an embodiment, alflutinib or a pharmaceutically acceptable salt thereof, is administered once daily.

In a further embodiment, alflutinib mesylate is administered once daily.

In an embodiment, the total daily dose of alflutinib mesylate is about 80 mg.

In an embodiment, the total daily dose of alflutinib mesylate is about 40 mg.

CX-101 (olafertinib; RX-518)

### CX-101 has the Following Chemical Structure:

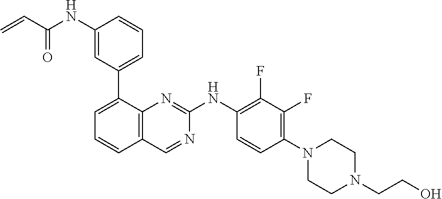 [SMILES: C=CC(=O)Nc1cccc(-c2cccc3cnc(Nc4ccc(N5CCN(CCO)CC5)c(F)c4F)nc23)c1]

The free base of CX-101 is known by the chemical name: N-(3-(2-((2,3-difluoro-4-(4-(2-hydroxyethyl)piperazin-1-yl)phenyl)amino)quinazolin-8-yl)phenyl)acrylamide. CX-101 is disclosed in WO 2015/027222. CX-101 is also known as RX-518 and olafertinib.

### HS-10296 (Almonertinib; Aumolertinib)

### HS-10296 (Almonertinib; Aumolertinib) has the Following Chemical Structure:

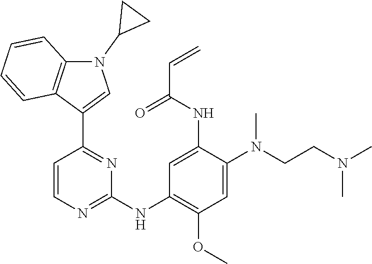 [SMILES: C=CC(=O)Nc1cc(Nc2nccc(-c3cn(C4CC4)c4ccccc34)n2)c(OC)cc1N(C)CCN(C)C]

The free base of HS-10296 is known by the chemical name: N-[5-[[4-(1-cyclopropylindol-3-yl)pyrimidin-2-yl]amino]-2-[2-(dimethylamino)ethyl-methyl-amino]-4-methoxy-phenyl]prop-2-enamide. HS-10296 is disclosed in WO 2016/054987.

In an embodiment, the total daily dose of HS-10296 is about 110 mg.

BPI-7711 (rezivertinib)

### BPI-7711 has the Following Chemical Structure:

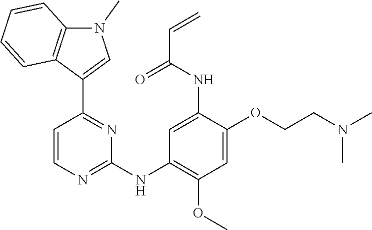 [SMILES: C=CC(=O)Nc1cc(Nc2nccc(-c3cn(C)c4ccccc34)n2)c(OC)cc1OCCN(C)C]

The free base of BPI-7711 is known by the chemical name: N-[2-[2-(dimethylamino)ethoxy]-4-methoxy-5-[[4-(1-methylindol-3-yl)pyrimidin-2-yl]amino]phenyl]prop-2-enamide. BPI-7711 is disclosed in WO 2016/94821.

In an embodiment, the total daily dose of BPI-7711 is about 180 mg.

Inhibitors of c-MET

In embodiments, the inhibitor of c-MET is any molecule which binds to and inhibits the activity of one or more c-MET (also known as mesenchymal epithelial transition factor) isoforms.

In embodiments, the inhibitor of c-MET is selected from the group consisting of savolitinib (Orpathys®; AZD6094; HMPL-504; volitinib) or a pharmaceutically acceptable salt thereof, capmatinib (Tabrecta®) or a pharmaceutically acceptable salt thereof, tepotinib (Tepmetko®) or a pharmaceutically acceptable salt thereof, glumetinib (SCC224) or a pharmaceutically acceptable salt thereof, and cabozantinib (Cometriq®, Cabometyx®) or a pharmaceutically acceptable salt thereof.

In embodiments, the inhibitor of c-MET is selected from the group consisting of savolitinib (Orpathys®; AZD6094; HMPL-504; volitinib) or a pharmaceutically acceptable salt thereof, capmatinib (Tabrecta®) or a pharmaceutically acceptable salt thereof, and tepotinib (Tepmetko®) or a pharmaceutically acceptable salt thereof.

In embodiments, the inhibitor of c-MET is savolitinib (Orpathys®; AZD6094; HMPL-504; volitinib) or a pharmaceutically acceptable salt thereof.

In embodiments, the inhibitor of c-MET is savolitinib (Orpathys®; AZD6094; HMPL-504; volitinib).

### Savolitinib (Orpathys®; AZD6094; HMPL-504; Volitinib)

### Savolitinib has the Following Chemical Structure:

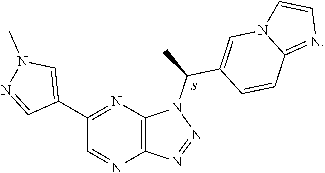

The free base of savolitinib is known by the chemical name 3-[(1S)-1-imidazo[1,2-a]pyridin-6-ylethyl]-5-(1-methylpyrrol-3-yl)triazolo[4,5-b]pyrazine. Savolitinib is described in WO2011079804 (compound 270).

In an embodiment, savolitinib, or a pharmaceutically acceptable salt thereof, is administered in the form of a pharmaceutical composition comprising one or more pharmaceutically acceptable excipients. In a further embodiment, the composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as magnesium stearate).

In an embodiment, the composition is in the form of a tablet.

In combinations with EGFR TKIs, such as osimertinib, savolitinib, or a pharmaceutically acceptable salt thereof, generally is administered to the subject at a daily dosage from about 100 mg to about 1200 mg.

In some embodiments, savolitinib, or a pharmaceutically acceptable salt thereof, is administered at a daily dosage from about 200 mg to about 800 mg. In one embodiment, savolitinib, or a pharmaceutically acceptable salt thereof, is administered at a daily dosage from about 300 mg to about 600 mg.

In some embodiments, savolitinib, or a pharmaceutically acceptable salt thereof, is administered to the subject once daily (QD). In some embodiments, savolitinib, or a pharmaceutically acceptable salt thereof, is administered to the subject twice daily (BID)

In one embodiment, savolitinib, or a pharmaceutically acceptable salt thereof, is administered at a dosage from about 300 mg to about 600 mg once daily.

In another embodiment, savolitinib, or a pharmaceutically acceptable salt thereof, is administered at a dosage of about 300 mg once daily. In another embodiment, savolitinib, or a pharmaceutically acceptable salt thereof, is administered at a dosage of about 600 mg once daily. In another embodiment, savolitinib, or a pharmaceutically acceptable salt thereof, is administered at a dosage of about 300 mg twice daily.

### Capmatinib (Tabrecta®)

### Capmatinib has the Following Chemical Structure:

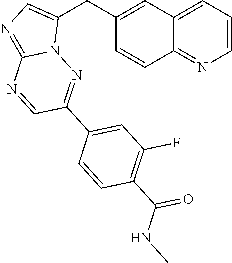 [SMILES: CNC(=O)c1ccc(-c2cnc3ncc(Cc4ccc5ncccc5c4)n3n2)cc1F]

Capmatinib is known by the chemical name 2-fluoro-N-methyl-4-[7-(quinolin-6-ylmethyl)imidazo[1,2-b][1,2,4]triazin-2-yl]benzamide. Capmatinib is disclosed in U.S. Pat. No. 7,767,675. In embodiments, capmatinib, or a pharmaceutically acceptable salt thereof, is administered once- or twice-daily. In further embodiments, capmatinib hydrochloride is administered twice-daily.

In embodiments, the total daily dose of capmatinib is about 800 mg (i.e. 400 mg twice daily). In embodiments, the total daily dose of capmatinib is about 600 mg (i.e. 300 mg twice daily). In embodiments, the total daily dose of capmatinib is about 400 mg (i.e. 200 mg twice daily).

### Tepotinib (Tepmetko®)

### Tepotinib has the Following Chemical Structure:

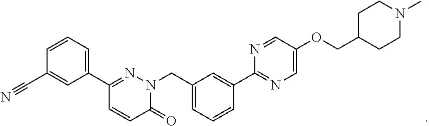 [SMILES: CN1CCC(COc2cnc(-c3cccc(Cn4nc(-c5cccc(C#N)c5)ccc4=O)c3)nc2)CC1]

The free base of tepotinib is known by the chemical name 3-[1-[[3-[5-[(1-methylpiperidin-4-yl)methoxy]pyrimidin-2-yl]phenyl]methyl]-6-oxopyridazin-3-yl]benzonitrile. Tepotinib is disclosed in U.S. Pat. No. 8,329,692. In embodiments, tepotinib, or a pharmaceutically acceptable salt thereof, is administered once- or twice-daily. In further embodiments, tepotinib hydrochloride hydrate is administered once-daily. In embodiments, the total daily dose of tepotinib is about 450 mg.

### Glumetinib (SCC224)

### Glumetinib has the Following Chemical Structure:

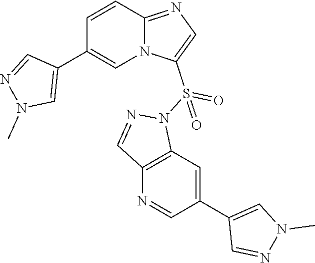 [SMILES: Cn1cc(-c2cnc3cnn(S(=O)(=O)c4cnc5ccc(-c6cnn(C)c6)cn45)c3c2)cn1]

The free base of glumetinib is known by the chemical name 6-(1-methyl-1H-pyrazol-4-yl)-1-((6-(1-methyl-1H-pyrazol-4-yl)imidazo(1,2-a)pyridin-3-yl)sulfonyl)-1H-pyrazolo(4,3-b)pyridine. Glumetinib is disclosed in WO2014201857. In embodiments, glumetinib, or a pharmaceutically acceptable salt thereof, is administered once- or twice-daily. In further embodiments, glumetinib is administered once-daily. In embodiments, the total daily dose of glumetinib is about 400 mg. In embodiments, the total daily dose of glumetinib is about 300 mg.

### Cabozantinib (Cometriq®, Cabometyx®)

### Cabozantinib has the Following Chemical Structure:

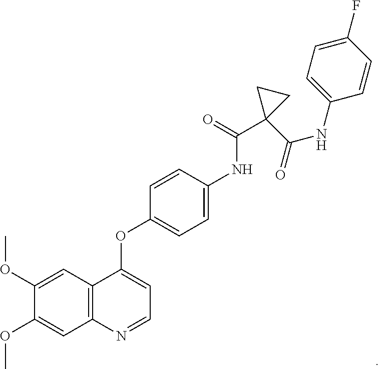 [SMILES: COc1cc2nccc(Oc3ccc(NC(=O)C4(C(=O)Nc5ccc(F)cc5)CC4)cc3)c2cc1OC]

The free base of cabozantinib is known by the chemical name N-(4-(6,7-dimethoxyquinolin-4-yloxy)phenyl)-N′-(4-fluorophenyl)cyclopropane-1,1-dicarboxamide, (2S)-hydroxybutanedioate. Cabozantinib is disclosed in U.S. Pat. No. 7,579,473. In embodiments, cabozantinib, or a pharmaceutically acceptable salt thereof, is administered once- or twice-daily. In further embodiments, cabozantinib (S)-malate is administered once-daily. In embodiments, the total daily dose of cabozantinib is about 60 mg.

### Further Embodiments

In an embodiment there is provided an EGFR TKI for use in the treatment of cancer in a human patient, wherein the EGFRTKI is administered in combination with an inhibitor of c-MET and wherein said cancer has a high or very high level of MET amplification and/or overexpression. In embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising administering to the human patient a therapeutically effective amount of an EGFR TKI, wherein the EGFR TKI is administered in combination with a therapeutically effective amount of an inhibitor of c-MET and wherein said cancer has a high or very high level of MET amplification and/or overexpression. In embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising administering to the human patient a first amount of an EGFR TKI, and a second amount of an inhibitor of c-MET, where the first amount and the second amount together comprise a therapeutically effective amount and wherein said cancer has a high or very high level of MET amplification and/or overexpression. In embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided the use of an EGFR TKI in the manufacture of a medicament for the treatment of cancer in a human patient, wherein the EGFR TKI is administered in combination with an inhibitor of c-MET and wherein said cancer has a high or very high level of MET amplification and/or overexpression. In embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a combination of an EGFR TKI and inhibitor of c-MET for use in the treatment of cancer in a human patient wherein said cancer has a high or very high level of MET amplification and/or overexpression. In embodiments the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising administering to the human patient a combination of a therapeutically effective amount of an EGFR TKI and a therapeutically effective amount of an inhibitor of c-MET wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising administering to the human patient a first amount of an EGFR TKI, and a second amount of an inhibitor of c-MET, where the first amount and the second amount together comprise a therapeutically effective amount and wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided the use of a combination of an EGFR TKI and inhibitor of c-MET in the manufacture of a medicament for treatment of cancer in a human patient, wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a combination of osimertinib or a pharmaceutically acceptable salt thereof and an inhibitor of c-MET for use in the treatment of cancer in a human patient, wherein the osimertinib, or pharmaceutically acceptable salt thereof, is administered to the human patient before the inhibitor of c-MET is administered to the human patient and wherein said cancer has a high or very high level of MET amplification and/or overexpression. In embodiments, the inhibitor of c-MET is savolitinib or a pharmaceutically acceptable salt thereof. In further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising administering to the human patient a combination of a therapeutically effective amount of osimertinib or a pharmaceutically acceptable salt thereof and a therapeutically effective amount of an inhibitor of c-MET, wherein the osimertinib, or pharmaceutically acceptable salt thereof, is administered to the human patient before the inhibitor of c-MET is administered to the human patient and wherein said cancer has a high or very high level of MET amplification and/or overexpression. In embodiments, the inhibitor of c-MET is savolitinib or a pharmaceutically acceptable salt thereof. In further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising administering to the human patient a first amount of osimertinib, or pharmaceutically acceptable salt thereof, and a second amount of an inhibitor of c-MET, where the first amount and the second amount together comprise a therapeutically effective amount, wherein the osimertinib, or pharmaceutically acceptable salt thereof, is administered to the human patient before the inhibitor of c-MET is administered to the human patient and wherein said cancer has a high or very high level of MET amplification and/or overexpression. In embodiments, the inhibitor of c-MET is savolitinib or a pharmaceutically acceptable salt thereof. In further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided the use of a combination of osimertinib or a pharmaceutically acceptable salt thereof and an inhibitor of c-MET for the manufacture of a medicament for the treatment of cancer in a human patient, wherein the osimertinib, or pharmaceutically acceptable salt thereof, is administered to the human patient before the inhibitor of c-MET is administered to the human patient and wherein said cancer has a high or very high level of MET amplification and/or overexpression. In embodiments, the inhibitor of c-MET is savolitinib or a pharmaceutically acceptable salt thereof. In further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided an EGFR TKI for use in the treatment of cancer in a human patient, wherein the treatment comprises the separate, sequential, or simultaneous administration of i) the EGFR TKI and ii) inhibitor of c-MET to the human patient and wherein said cancer has a high or very high level of MET amplification and/or overexpression. Where treatment is separate or sequential, the interval between the dose of EGFRTKI and the dose of inhibitor of c-MET may be chosen to ensure the production of a combined therapeutic effect.

A “therapeutic effect” encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

In embodiments, the administration of the EGFR TKI and the inhibitor of c-MET is sequential and the EGFR TKI is administered prior to the inhibitor of c-MET.

In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising the separate, sequential, or simultaneous administration of i) a therapeutically effective amount of an EGFR TKI and ii) a therapeutically effective amount of an inhibitor of c-MET to the human patient, wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating cancer in a human patient in need of such a treatment comprising the separate, sequential, or simultaneous administration of i) a first amount of an EGFR TKI and ii) a second amount of an inhibitor of c-MET to the human patient, where the first amount and the second amount together comprise a therapeutically effective amount and wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided use of an EGFR TKI in the manufacture of a medicament for the treatment of cancer in a human patient, wherein the treatment comprises the separate, sequential, or simultaneous administration of i) the EGFR TKI and ii) inhibitor of c-MET to the human patient and wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided an inhibitor of c-MET for use in the treatment of cancer in a human patient, wherein the inhibitor of c-MET is administered in combination with an EGFR TKI and wherein said cancer has a high or very high level of MET amplification and/or overexpression.

In an embodiment there is provided an inhibitor of c-MET for use in the treatment of cancer in a human patient, wherein the treatment comprises the separate, sequential, or simultaneous administration of i) an inhibitor of c-MET and ii) an EGFR TKI to the human patient and wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided use of an inhibitor of c-MET in the manufacture of a medicament for the treatment of cancer in a human patient, wherein the treatment comprises the separate, sequential, or simultaneous administration of i) an EGFR TKI and ii) the inhibitor of c-MET to the human patient and wherein said cancer has a high or very high level of MET amplification and/or overexpression. In embodiments, the EGFR TKI is osimertinib or a pharmaceutically acceptable salt thereof. In further embodiments, the inhibitor of c-MET is savolitinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is EGFR mutation-positive NSCLC. In yet further embodiments, the high or very high level of MET amplification and/or overexpression is defined by FISH10+ and/or IHC90+.

In an embodiment there is provided a method of treating locally advanced or metastatic NSCLC in a human patient in need of such a treatment comprising administering to the human patient a therapeutically effective amount of osimertinib, or a pharmaceutically acceptable salt thereof, wherein the osimertinib, or a pharmaceutically acceptable salt thereof, is administered in combination with savolitinib, wherein said locally advanced or metastatic NSCLC has a high or very high level of MET amplification and/or overexpression defined by FISH6+, FISH7+, FISH8+, FISH9+ or FISH10+ and/or IHC60+, IHC70+, IHC80+ or IHC90+, and wherein said human patient's locally advanced or metastatic NSCLC has progressed during or after previous treatment with osimertinib, or a pharmaceutically acceptable salt thereof.

In an embodiment there is provided a method of treating locally advanced or metastatic NSCLC in a human patient in need of such a treatment comprising administering to the human patient a therapeutically effective amount of savolitinib, wherein the savolitinib is administered in combination with osimertinib, or a pharmaceutically acceptable salt thereof, wherein said locally advanced or metastatic NSCLC has a high or very high level of MET amplification and/or overexpression defined by FISH6+, FISH7+, FISH8+, FISH9+ or FISH10+ and/or IHC60+, IHC70+, IHC80+ or IHC90+, and wherein said human patient's locally advanced or metastatic NSCLC has progressed during or after previous treatment with osimertinib, or a pharmaceutically acceptable salt thereof.

In an embodiment there is provided a kit comprising:
a first pharmaceutical composition comprising an EGFR TKI and a pharmaceutically acceptable excipient; and
a second pharmaceutical composition comprising an inhibitor of c-MET and a pharmaceutically acceptable excipient.

### Examples

The specific Examples below, with reference to the accompanying Figures, are provided for illustrative purposes only and are not to be construed as limiting the teachings herein.

### Study D5084C00007 (SAVANNAH)

The combination of osimertinib (80 mg QD) with savolitinib (300 mg QD, 300 mg BID, or 600 mg QD) is being investigated in the ongoing Phase 2 Study D5084C00007 (SAVANNAH) to understand the ability of the combination to overcome MET amplification and/or MET overexpression as a mechanism of resistance in patients with locally advanced or metastatic EGFRm+ NSCLC who had experienced disease progression following osimertinib therapy.

Identification of MET amplification and/or overexpression status in the SAVANNAH study is based primarily on prospective central testing of tumor biopsies collected after progression on prior treatment with osimertinib using 2 assays: MET FISH (Vysis MET FISH Probe Kit) and MET IHC (VENTANA MET (SP44) RxDx Assay). A positive MET result by either or both of the two assays makes a patient eligible for screening for enrolment in the trial. Per inclusion criteria, patients are allowed to have received up to 3 lines of prior therapy, which must include osimertinib as one of the prior lines of therapy, but could also include other EGFR TKIs, chemotherapy, or chemotherapy in combination with an 10 agent in the metastatic setting. The primary objective of this study is to determine efficacy.

Approximately 259 patients are planned to be enrolled and treated with osimertinib 80 mg QD in combination with either savolitinib 300 mg QD (approximately n=196), Savolitinib 300 mg BID (approximately n=33), or savolitinib 600 mg QD (approximately n=33) in this study. although SAVANNAH is currently ongoing, patient recruitment into the 300 mg QD dosing cohort has completed.

As of a data cut-off (DCO) of June 2021, a total of 253 patients had received at least one dose of study treatment on SAVANNAH. Of these 253 patients, 196 patients (87 2 L patients [44.4%] and 109≥3 L patients [55.6%]) have received treatment with a starting dose of 300 mg QD, in combination with osimertinib (80 mg QD). Thirty-six patients (18.4%) had prior platinum-based chemotherapy and 12 patients (6%) had prior PD-(L)1 therapy. Furthermore, at this DCO, 27 patients (17 2 L patients and 10≥3 L patients), and 30 patients (8 2 L patients and 22≥3 L patients) have received treatment with a starting dose of 300 mg BID and 600 mg QD savolitinib, respectively, in combination with osimertinib (80 mg QD).

A total of 108 patients have received at least one dose of savolitinib 300 mg QD plus osimertinib 80 mg QD and had high MET level status (IHC90+ and/or FISH10+). Thirty-five patients (32%) had recurrent disease and 38 patients (35%) had brain metastases at study entry. Fifty patients (46%) received 2 L treatment, 37 patients (34%) received 3 L treatment without prior chemotherapy in the metastatic setting, and 21 patients (19.4%) received 3 L treatment with prior chemotherapy in the metastatic setting. Fifty-five patients (50.9%) had one prior line of EGFR TKI and 51 patients (47.2%) had two prior lines of EGFR TKIs. Ninety-nine patients (92%) had osimertinib as their immediately prior line of therapy at study entry.

At the second interim analysis (DCO of October 2020), 137 patients (33 2 L patients and 104≥3 L patients) had received treatment with a starting dose of 300 mg QD in combination with osimertinib and had the opportunity to have at least 2 post baseline Response Evaluation Criteria In Solid Tumors (RECIST) scans. The confirmed objective response rate (ORR) and duration of response (DoR) results from these patients are summarized in Table 1.

TABLE 1

| | 2L Patients | ≥3L Patients | All |
|---|---|---|---|
| | (N = 33) | (N = 104) | (N = 137) |
| ORR, % (95% CI) | 36 | 26 | 29 |
| | (20.4, 54.9) | (17.9, 35.5) | (21.1, 36.8) |
| Median DoR in months | NC | 6.08 | 6.41 |

Biomarker cut-off was FISH5+ and/or IHC50+;
CI is confidence interval

An exploratory analysis was performed in these 137 patients who progressed during or following osimertinib to better understand the relationship between levels of MET amplification and/or overexpression and efficacy. Analysis of ORR and median progression free survival (PFS) based on MET overexpression level as detected by central MET IHC and FISH assays (Tables 2 and 3) showed a trend toward an improved response rate with increasing level of MET overexpression and amplification. Therefore, ≥10 copies of MET gene (referred to as FISH10+) and ≥90% of tumor cells staining at 3+ intensity (referred to as IHC9+) was selected as the provisional optimal cut-off for MET FISH and MET IHC.

TABLE 2

| % of tumor | | | | | | |
|---|---|---|---|---|---|---|
| cells with 3+ | Patients with MET overexpression | | | Patients without MET overexpression | | |
| staining | status meeting cut-off | | | status meeting cut-off | | |
| intensity | | Median PFS | | | Median PFS | |
| used as cut- | | (95% CI) | ORR, % | | (95% CI) | ORR, % |
| off (N = 132)^{a} | N | (events) | (n) | N | (events) | (n) |
| IHC50+^{b} | 118 | 5.5 (4.0, 7.2) | 29 | 14 | 2.6 (1.4, NA) | 7 |
| | | (69/118) | (34/118) | | (10/14) | (1/14) |
| 60 | 99 | 5.8 (4.2, 7.3) | 32 | 33 | 2.6 (1.5, 4.7) | 9 |
| | | (54/99) | (32/99) | | (25/33) | (3/33) |
| 70 | 86 | 5.8 (4.2, 7.3) | 36 | 46 | 2.9 (1.5, 5.8) | 9 |
| | | (47/86) | (31/86) | | (32/46) | (4/46) |
| 80 | 74 | 5.8 (4.4, NA) | 41 | 58 | 2.9 (1.7, 4.5) | 9 |
| | | (40/74) | (30/74) | | (39/58) | (5/58) |
| 90 | 64 | 5.8 (4.4, NA) | 42 | 68 | 3.0 (2.6, 4.7) | 12 |
| | | (35/64) | (27/64) | | (44/68) | (8/68) |
| 100 | 52 | 5.8 (4.4, NA) | 44 | 80 | 3.9 (2.7, 5.8) | 15 |
| | | (27/52) | (23/52) | | (52/80) | (12/80) |

^{a}132 patients had valid central laboratory MET IHC results available for analysis
^{b}IHC50+ indicates cut-off ≥50% tumor cells staining with strong 3+ intensity as used for patient selection onto the SAVANNAH study

TABLE 3

| | Patients with MET amplification | | | Patients without MET amplification | | |
|---|---|---|---|---|---|---|
| | status meeting cut-off | | | status meeting cut-off | | |
| MET gene copy | | Median PFS | | | Median PFS | |
| number used as | | (95% CI) | ORR, % | | (95% CI) | ORR, % |
| cut-off (N = 130)^{a} | N | (events) | (n) | N | (events) | (n) |
| FISH5+^{b} | 88 | 5.5 (4.1, | 35 | 42 | 4.2 (2.7, | 17 |
| | | 7.2) | (31/88) | | 9.0) | (7/42) |
| | | (48/88) | | | (29/42) | |
| 6 | 76 | 5.6 (4.3, | 38 | 54 | 4.2 (2.7, | 17 |
| | | 7.3) | (29/76) | | 7.3) | (9/54) |
| | | (41/76) | | | (36/54) | |
| 7 | 68 | 5.6 (4.3, | 41 | 62 | 4.2 (2.7, | 16 |
| | | 7.2) | (28/68) | | 7.3) | (10/62) |
| | | (37/68) | | | (40/62) | |
| 8 | 59 | 5.6 (4.2, | 42 | 71 | 4.2 (2.8, | 18 |
| | | 7.2) | (25/59) | | 7.3) | (13/71) |
| | | (33/59) | | | (44/71) | |
| 9 | 49 | 5.6 (4.4, | 45 | 81 | 4.2 (2.7, | 20 |
| | | 7.3) | (22/49) | | 7.3) | (16/81) |
| | | (27/49) | | | (50/81) | |
| 10 | 41 | 5.8 (5.5, | 49 | 89 | 4.1 (2.8, | 20 |
| | | NA) | (20/41) | | 6.7) | (18/89) |
| | | (20/41) | | | (57/89) | |
| 11 | 37 | 5.8 (4.4, | 49 | 93 | 4.2 (2.9, | 22 |
| | | NA) | (18/37) | | 6.7) | (20/93) |
| | | (19/37) | | | (58/93) | |
| 12 | 33 | 5.6 (4.3, | 45 | 97 | 4.5 (2.9, | 24 |
| | | NA) | (15/33) | | 7.3) | (23/97) |
| | | (18/33) | | | (59/97) | |
| 13 | 29 | 5.6 (4.3, | 45 | 101 | 4.5 (3.0, | 25 |
| | | NA) | (13/29) | | 7.3) | (25/101) |
| | | (17/29) | | | (60/101) | |

^{a}130 patients had valid central laboratory MET FISH results available for analysis
^{b}FISH5+ indicates cut-off of MET gene copy number ≥5 or MET:CEP7 ration ≥2 (%) as used for patient selection onto the SAVANNAH study [CEP7 is centromere 7]

Subsequently, the efficacy in the high biomarker groups (FISH10+ and/or IHC90+) was further evaluated in a larger number of patients with longer follow up, who had received treatment with a starting dose of 300 mg savolitinib QD in combination with osimertinib and had the opportunity to have at least 2 post baseline RECIST scans (DCO of June 2021; N=193). Within this population, 108 patients met the criteria of FISH10+ and/or IHC90+ status. As summarized in Table 4, efficacy in patients with FISH10+ and/or IHC90+ positive status (ORR 49.1%, DoR 9.6 months, PFS 7.1 months) was improved compared to all patients (ORR 32.1%, DoR 8.3 months, PFS 5.3 months), and particularly in comparison to the subgroup without FISH10+ and without IHC90+ status (ORR 9.1%, DoR 6.9 months, PFS 2.8 months).

TABLE 4

| | | Population | Population |
|---|---|---|---|
| | | with FISH10+ | without FISH10+ |
| | | and/or IHC90+ | and without |
| | All patients^{a} | status^{b} | IHC90+ status^{c} |
| | (N = 193) | (N = 108) | (N = 77) |
| ORR, % (n) (95% CI) | 32.1 (62) | 49.1 (53) | 9.1 (7) |
| | (25.6, 39.2) | (39.3, 58.9) | (3.7, 17.8) |
| Median DoR, months | 8.3 | 9.6 | 6.9 |
| (95% CI) | (6.9, 10.6) | (6.6, 10.6) | (4.1, 16.9) |
| Percent of responders | 46.8% (29) | 50.9% (27) | 14.3% (1) |
| still in response at | | | |
| DCO (n) | | | |
| Median PFS, months | 5.3 | 7.1 | 2.8 |
| (95% CI) | (4.2, 5.8) | (5.3, 9.2) | (2.6, 4.3) |

^{a}Patients with FISH5+ and/or IHC50+ status who had received treatment with a starting dose of 300 mg QD in combination with osimertinib and had opportunity to have at least 2 post baseline RECIST scans. Includes 8 patients which are excluded in ^{b} and ^{c} due to invalid or missing test result
^{b}Subgroup of patients with FISH10+ and/or IHC90+ status based on exploratory analysis of central laboratory data.
^{c}Patients positive for eligibility cut-off but without FISH10+ and IHC90+ status based on exploratory analysis of central laboratory data

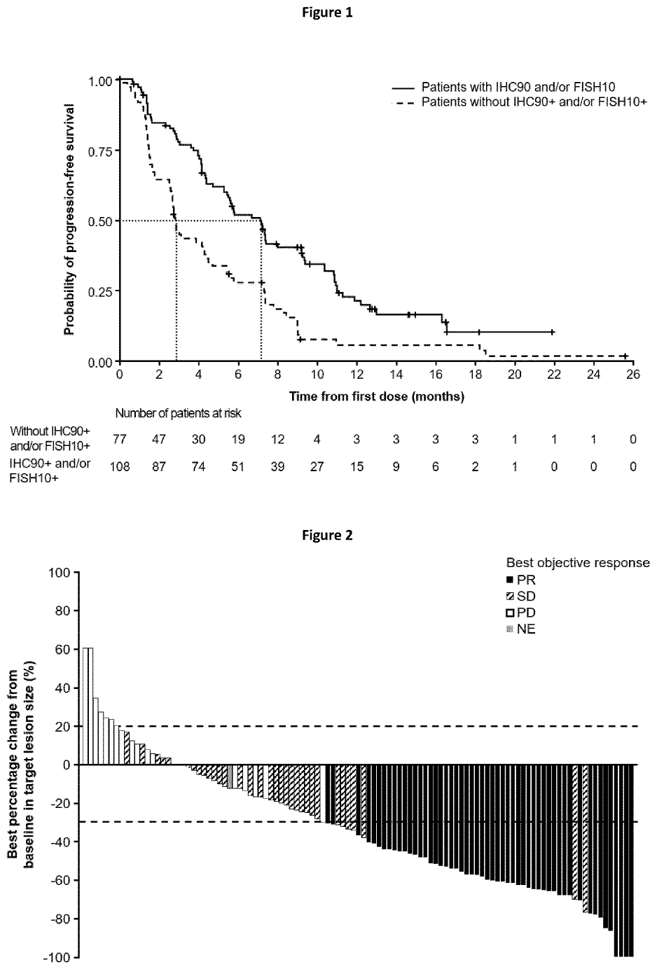

Furthermore, in terms of PFS, FIG. 1 shows good separation of the Kaplan-Meier curves between the patients with FISH10+ and/or IHC90+ status, and the patients without FISH10+ and/or IHC90+ status, further supporting the optimal cut-off to identify the population to treat.

As shown in Table 5, within the FISH10+ and/or IHC90+ population, an ORR of 60.0% (95% Cl 45.2, 73.6) was observed in the 2 L patients (N=50), an ORR of 40.5% (95% Cl 24.8, 57.9) was observed in ≥3 L patients without previous chemotherapy (N=37), and an ORR of 38.1% (95% Cl 18.1, 61.6) was observed in ≥3 L patients with previous chemotherapy (N=21). Median DoR was 9.6 months in the 2 L patients, 10.6 months in the ≥3 L patients without previous chemotherapy, and 7.2 months in the ≥3 L patients with chemotherapy.

TABLE 5

| | | | ≥3L (no | ≥3L |
|---|---|---|---|---|
| | All patients | 2L patients | chemotherapy) | (chemotherapy) |
| | N = 108 | N = 50 | N = 37 | N = 21 |
| ORR, % (n) | 49.1 (53) | 60.0 (30) | 40.5 (15) | 38.1 (8) |
| (95% CI) | (39.3, 58.9) | (45.2, 73.6) | (24.8, 57.9) | (18.1, 61.6) |
| Median DoR, | 9.6 | 9.6 | 10.6 | 7.2 |
| months (95% CI) | (6.6, 10.6) | (6.0, NC) | (5.8, NC) | (2.8, 9.3) |
| Percentage of | 50.9% (27) | 66.7% (20) | 46.7% (7) | 0 |
| responders still in | | | | |
| response at DCO (n) | | | | |
| Median PFS, months | 7.1 | 7.4 | 6.7 | 5.8 |
| (95% CI) | (5.3, 9.2) | (4.7, 11.0) | (3.8, 9.2) | (4.2, 10.9) |

Tumour response is shown in FIG. 2, which is a waterfall plot showing the best percentage change in target lesion in patients with FISH10+ and/or IHC90+. Evaluable for efficacy set was defined as dosed patients with measurable disease at baseline who had ≥2 on-treatment RECIST scans. FISH10+, fluorescence in situ hybridisation (MET copy number ≥10); IHC90+, 3+ immunohistochemistry overexpression in 90% of tumour cells; NE, not evaluable; PD, progressive disease; PR, partial response; SD, stable disease.

1-17. (canceled)

18. A method of treating cancer in a human patient in need of such a treatment, comprising administering to the human patient a first amount of an EGFR TKI, and a second amount of an inhibitor of c-MIET, where the first amount and the second amount together comprise a therapeutically effective amount, and wherein said cancer has a high or very high level of MET amplification and/or overexpression defined by FISH6+, FISH7+, FISH8+, FISH9+ or FISH10+ and/or IHC60+, IHC70+, IHC80+ or IHC90+.

19-21. (canceled)

22. The method of claim 18, wherein the administration of the EGFR TKI and the inhibitor of c-MET is separate, sequential, or simultaneous.

23. The method of claim 18, wherein the high or very high level of MET amplification and/or overexpression is defined by FISH10+ and/or IHC90+.

24. The method of claim 18, wherein the EGFR TKI is a compound of Formula (I): 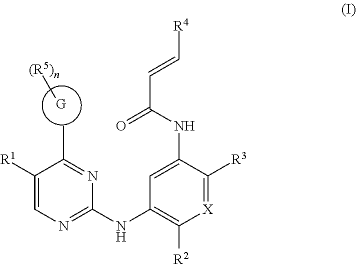
wherein:
G is selected from 4,5,6,7-tetrahydropyrazolo[1,5-a]pyridin-3-yl, indol-3-yl, indazol-1_-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;
R¹ is selected from hydrogen, fluoro, chloro, methyl and cyano;
R² is selected from methoxy, trifluoromethoxy, ethoxy, 2,2,2-trifluoroethoxy and methyl;
R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperazin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;
R⁴ is selected from hydrogen, 1-piperidinomethyl and N,N-dimethylaminomethyl;
R⁵ is independently selected from methyl, ethyl, propyl, 2,2-difluoroethyl, 2,2,2-trifluoroethyl, fluoro, chloro and cyclopropyl;
X is CH or N; and
n is 0, 1 or 2;
or a pharmaceutically acceptable salt thereof.

25. The method of claim 24, wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from [2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

26. The method of claim 18, wherein the EGFR TKI is selected from the group consisting of osimertinib or a pharmaceutically acceptable salt thereof, AZD3759 or a pharmaceutically acceptable salt thereof, alflutinib or a pharmaceutically acceptable salt thereof, HS-10296 or a pharmaceutically acceptable salt thereof, and lazertinib or a pharmaceutically acceptable salt thereof.

27. The method of claim 18, wherein the EGFR TKI is osimertinib or a pharmaceutically acceptable salt thereof.

28. The method of claim 18, wherein the inhibitor of c-MET is selected from the group consisting of savolitinib or a pharmaceutically acceptable salt thereof, capmatinib or a pharmaceutically acceptable salt thereof, and tepotinib or a pharmaceutically acceptable salt thereof.

29. The method of claim 28, wherein the inhibitor of c-MET is savolitinib or a pharmaceutically acceptable salt thereof.

30. The method of claim 29, wherein the savolitinib, or a pharmaceutically acceptable salt thereof, is administered at a dosage of about 600 mg once daily or about 300 mg twice daily or about 300 mg once daily.

31. The method of claim 18, wherein the cancer is EGFR mutation-positive non-small cell lung cancer.

32. The method of claim 31, wherein the EGFR mutation-positive non-small cell lung cancer comprises an activating mutation in EGFR selected from exon 19 deletions and L858R substitution mutations.

33. The method of claim 18, wherein the human patient's cancer has progressed during or after previous treatment with osimertinib.
